(19) United States
(12) Patent Application Publication
(10) Pub. No.: US 2026/0257048 A1
(43) Pub. Date: Sep. 3, 2026

(54) Biodegradable Implantable Drug Delivery Device with Barb-Based Retention Mechanism

(71) Applicant: Mark T. McBride, Coronado, CA (US)

(72) Inventor: Mark T. McBride, Coronado, CA (US)

(21) Appl. No.: 19/550,415

(22) Filed: Feb. 26, 2026

Related U.S. Application Data
(60) Provisional application No. 63/765,858, filed on Mar. 3, 2025.

(51) Int. Cl. A61M 31/00

(52) U.S. Cl. CPC A61M 31/002

(57) ABSTRACT

This invention provides a novel, biodegradable implantable drug delivery system that securely remains in place for a predetermined duration before allowing for minimally invasive removal. By utilizing biodegradable materials and a barb-based retention mechanism, the device addresses key challenges in localized pain management, improving patient outcomes while reducing complications associated with traditional implanted catheters.

### CROSS-REFERENCE TO RELATED APPLICATION

This application claims benefit of U.S. Patent Application 63/765,858, filed on Mar. 3, 2025, the disclosure of which is hereby incorporated by reference in its entirety to provide continuity of disclosure to the extent such a disclosure is not inconsistent with the disclosure herein.

### FIELD OF THE INVENTION

The present invention relates to implantable medical devices; specifically biodegradable drug delivery implants designed for localized medication administration. More particularly, the invention is related to a biodegradable catheter-like device having an anti-microbial coating that may located over a layer of a biodegradable coating that includes biodegradable barbed structures formed in the biodegradable coating layer that can be utilized to determine a retention duration of the catheter-like device and facilitate controlled removal as the biodegradable barbed structures degrade. In this manner, the biodegradable barbed structures assist in retaining a catheter tip in a proper position until it is time for the catheter to be removed, at which time enough of the biodegradable barbed structures have been absorbed into the patient's body to allow the catheter to be easily withdrawn.

### BACKGROUND OF THE INVENTION

Effective localized drug delivery is critical in both acute and chronic pain management. Traditional catheters and implanted drug delivery systems often require manual removal, which can lead to infection, mechanical failure, or discomfort. Therefore, there is a need for a drug delivery device that can be implanted for a defined period, remain securely in place, and degrade in a controlled manner to facilitate easy removal.

### SUMMARY OF THE INVENTION

The present invention is an implantable, biodegradable medical device designed to deliver medication, such as local anesthetics or analgesics, to targeted areas of the body for pain management. The device is primarily intended for short-term and long-term pain relief applications, including post-surgical pain control and palliative care.

The device consists of a catheter-like structure composed of non-biodegradable materials (such as polypropylene, nylon, or polyester) which allows for liquid medications such as anesthetic to be released through small fenestrations in the distal end of the catheter-like structure. The external surface of the device is coated with an anti-microbial coating that may be located over a layer of an absorbent (i.e., biodegradable) materials, wherein the absorbent layer also includes barbed structures formed into the absorbent layer where the duration of catheter stabilization can be modified based on application by having different lengths and/or shapes of the biodegradable barbed structures:
1.) Longer barbs provide extended retention before degradation facilitates removal; or
2.) Shorter barbs degrade more quickly, allowing for earlier removal of the device.

As the biodegradable barbed structures degrade over time, the device can be more easily extracted with minimal tissue disruption. The device is designed to be connected to a drug pump, which delivers medication through the implant.

### BRIEF DESCRIPTION OF THE DRAWINGS

Some embodiments of the present invention are illustrated as an example and are not limited by the figures of the accompanying drawings, in which like references may indicate similar elements and in which:

FIG. 1 is a schematic illustration of a biodegradable implantable drug delivery device with a barb-based retention mechanism, constructed according to various embodiments of the present invention described herein; and

FIG. 2 is a schematic illustration of the catheter showing a close-up view of the coating having the biodegradable barbed structures, constructed according to various embodiments of the present invention described herein;

FIG. 3 is a schematic cross-sectional view taken along lines 3-3 in FIG. 2 that shows the coating on the drug delivery device on the drug delivery device and the biodegradable barbed structures in the coating, constructed according to various embodiments of the present invention described herein;

FIG. 4 is a schematic cross-sectional view taken along lines 4-4 in FIG. 1 that shows the fenestrations in the end of the drug delivery device that are configured to allow the liquid medications such as an anesthetic to be dispensed from the drug delivery device, constructed according to various embodiments of the present invention described herein; and

FIG. 5 is a cross-sectional view taken from circle A in FIG. 1 that shows the construction of the barbed structure and an optional anti-microbial layer, constructed according to various embodiments of the present invention described herein.

### DETAILED DESCRIPTION OF THE INVENTION

The terminology used herein is to describe particular embodiments only and is not intended to be limiting to the invention. As used herein, the term “and/or” includes any and all combinations of one or more of the associated listed items. As used herein, singular forms “a”, “an”, and “the” are intended to include the plural forms as well as the singular forms unless the context clearly indicates otherwise. It will be further understood that the terms “comprises” and/or “comprising,” when used in this specification, specify the presence of stated features, steps, operations, elements, and/or components but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefits, and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

A new and unique biodegradable implantable drug delivery device with an absorbable (i.e., biodegradable) coating layer having a biodegradable barbed structure retention mechanism and an optional anti-microbial coating being located over the biodegradable coating layer is discussed herein. In the following description, for purposes of explanation, numerous specific details are set forth to provide a thorough understanding of the present invention. It will be evident, however, to one skilled in the art that the present invention may be practiced without these specific details.

The present disclosure is considered an exemplification of the invention and is not intended to limit the invention to the specific embodiments illustrated by the figures or description below.

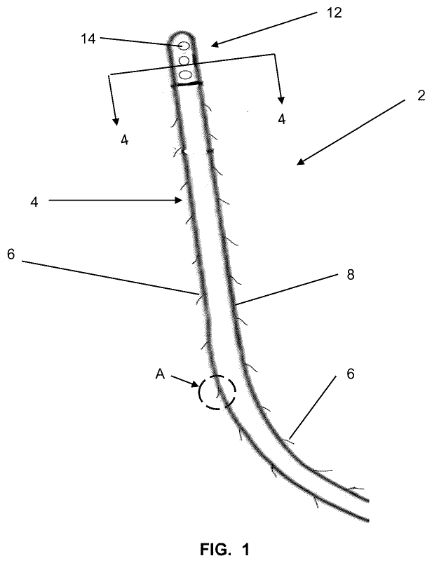

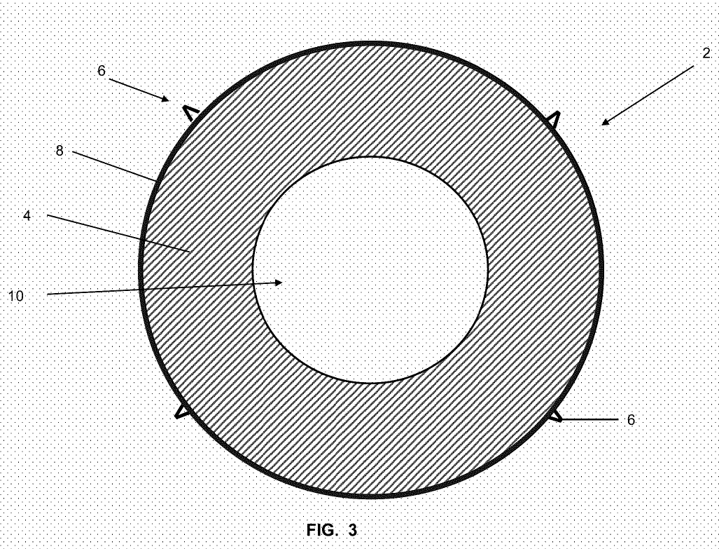

The present invention will now be described by referencing the appended figures representing preferred embodiments. With respect to FIGS. 1-5, there is illustrated a biodegradable implantable drug delivery device 2 with an optional anti-microbial coating 9 that may be located over a biodegradable coating layer 8 having biodegradable barbed structures 6 that can be utilized as a drug delivery device retention mechanism.

### 1. Device Structure

Catheter-Like Body: As show in FIG. 1, the drug delivery device 2 includes, in part, a tubular structure 4 formed from non-biodegradable polymers such as (such as polypropylene, nylon, or polyester). The material composition is selected to provide mechanical integrity for a predetermined period.

Biodegradable Barbed Retention Mechanism: As shown in FIGS. 1-5, the outer surface of the tubular structure 4 of device 2 includes an optional anti-microbial coating 9 that may be located over an absorbable, anti-microbial coating layer 8 composed of absorbent (biodegradable) materials that is proximal to the tubular structure 4 such that the coating 8 includes biodegradable barbed structures 6 formed within the biodegradable coating layer 8 where the duration of catheter stabilization can be modified based on application by having different compositions, lengths, and/or shapes of the biodegradable barbed structures 6:
1.) Longer barbs provide extended retention before degradation facilitates removal; or
2.) Shorter barbs degrade more quickly, allowing for earlier removal of the device.

A unique aspect of the present invention is that the optional anti-microbial coating that may be located over the biodegradable coating layer 8 and the barbed structures 6 formed in the biodegradable coating 8. In particular, the optional anti-microbial coating 9 is conventionally applied over the biodegradable layer 8 and the barbed structures in order to reduce the likelihood of infection from the device 2 while the device 2 is retained within a patient.

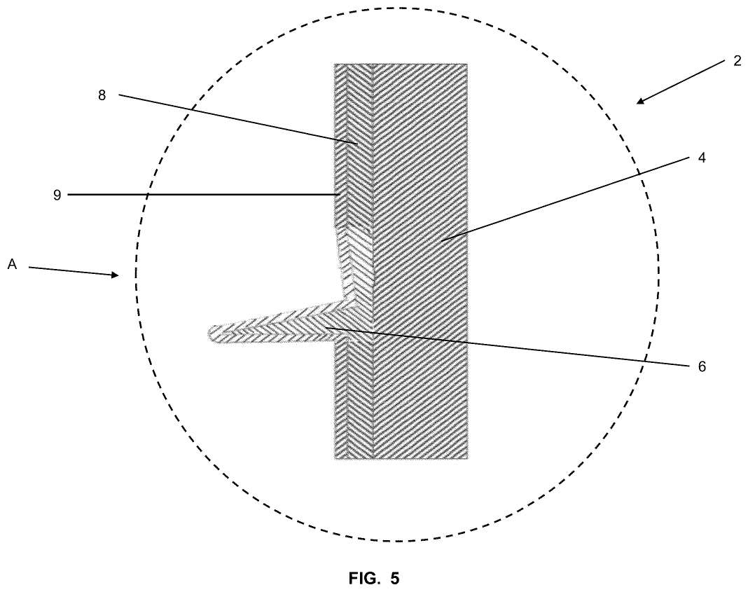

Another unique aspect of the present invention is the use of the biodegradable coating layer 8. In particular, the biodegradable coating layer 8 is applied to the outer surface of the tubular structure 4 of device 2. The biodegradable coating layer 8 is then conventionally dried. Once the biodegradable coating layer 8 is dried, the barbed structures 6 are formed in the biodegradable coating layer 8 around the periphery of the tubular structure 4 by conventional techniques such as cutting, forming, or the like. It is to be understood that the thickness and/or the depth at which the barbed structures 6 are formed in the biodegradable coating layer 8 determines the length of time in which the device 2 can be retained within the patient, as will be described in greater detail later. After the barbed structures 6 are formed in the biodegradable coating 8, the optional anti-microbial coating 9 may be applied to the biodegradable coating layer 8 and the barbed structures 6 (FIG. 5) A still another unique aspect of the present invention is that the coating layer 8 and the barbed structures 6 should degrade at a controlled rate. It is to be further understood that the coating 8 can be formulated so that the size of the barbed structures 6 and the degree at which the coating layer 8 (and barbed structures 6) degrade can be adjusted depending upon how long the drug delivery device 2 is to be retained within the patient. For example, the size of the barbed structures 6 that are formed in the coating 8 can be increased if it is desired for the drug delivery device 2 to be retained within the patient for a longer period of time. Conversely, the size of the barbed structures 6 that are formed in the coating layer 8 can be decreased if it is desired for the drug delivery device 2 to be retained within the patient for a shorter period of time. Furthermore, the coating layer 8 can be adjusted (i.e., increased coating thickness and/or coating composition) so that the rate of the controlled degradation of the coating layer 8 is slower if it is desired for the drug delivery device 2 to be retained within the patient for a longer period. Conversely, the coating layer 8 can be adjusted (i.e., decreased coating thickness and/or coating composition) so that the rate of the controlled degradation of the coating layer 8 is quicker if it is desired for the drug delivery device 2 to be retained within the patient for a shorter period of time. A key feature of the present invention being that the length and thickness of the barbed structures 6 and/or the thickness and composition of coating layer 8 determine how long the device 2 can remain implanted in the patient.

A unique aspect of the present invention is that, in one embodiment, once the biodegradable barbed structures 6 sufficiently degrade, the device 2 can be removed with reduced resistance.

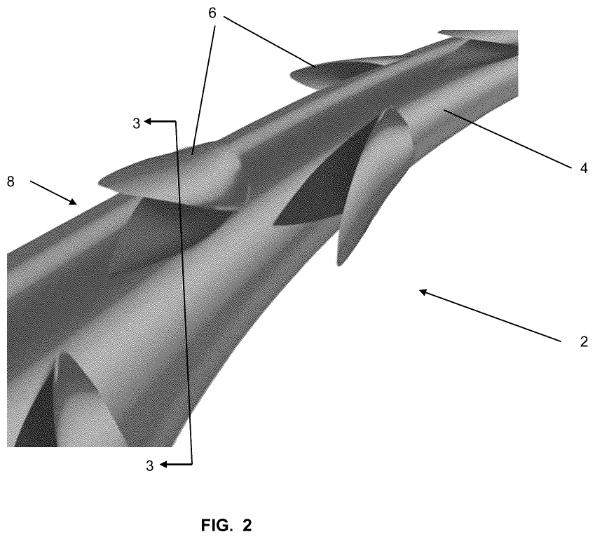

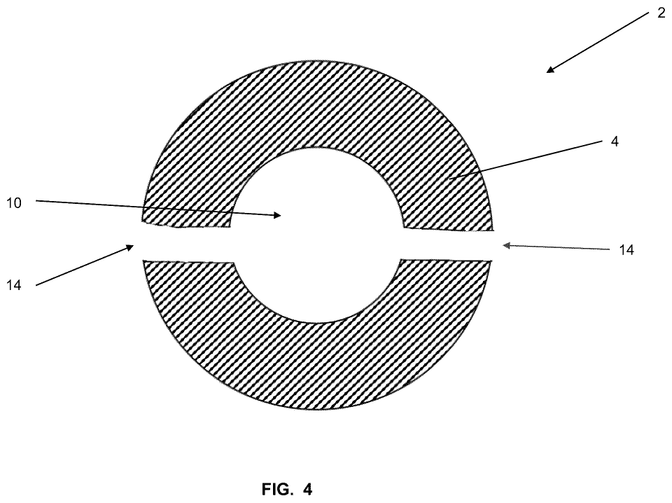

Drug Delivery Pathway: As shown in FIGS. 2-4, the non-biodegradable tubular structure 4 of implantable drug delivery device 2 also includes a smooth area 12 located at the end of the tubular structure 4 of device 2, wherein the smooth area 12 includes fenestrations or holes 14 that allow drug elution (i.e., drug delivery). It is to be understood that the smooth area 12 is not coated by the biodegradable coating layer 8 or the optional anti-microbial layer 9. It is also to be understood that the medications are delivered to the patient through the holes 14. As shown in FIG. 4, a lumen 10 (i.e., an open space) located within a length of the tubular structure 4 of device 2. It is to be understood that the lumen 10 is configured to be able to assist in delivering medication along an interior of the tubular structure 4 to the smooth area 12 where the medication can then be delivered to the patient through holes 14.

Connection to Drug Pump: It is to be understood that the device 2 can designed to interface with an external or implantable drug pump (not shown), thereby ensuring precise and continuous drug administration.

### 2. Functional Use Cases

Acute Pain Management: The device 2 can be implanted near nerves to deliver local liquid medications such as local anesthetics for post-surgical pain relief. A shorter design of the biodegradable barbed structures 6 allows for removal within days. In this manner, the device 2 can be configured to deliver local anesthetics for short-term pain management

Chronic Pain Management: For palliative care and long-term pain control, the device 2 can be used with a slow-degrading design of the biodegradable barbed structures 6, thereby remaining in place for weeks or months before removal is necessary. In this manner, the device 2 can be configured for long-term drug delivery for chronic pain management.

Customizable Retention Duration: The biodegradable barbed structure 6 allows for tailored retention periods depending on patient needs and treatment plans. In particular, a determination is made as to how long the device 2 should be retained within the patient. The formulation of the coating layer 8 is then determined so that the degradation of the coating layer 8 and, more importantly, the barbed structures 6 can be controlled in order for the device 2 to be retained within the patient for the desired period of time, but when the time period ends, the barbed structures 6 (and somewhat coating layer 8) have degraded sufficiently to allow the device 2 to be easily and quicky removed from the patient without creating any unnecessary patient discomfort. The device 2 is then conventionally inserted into the patient at the desired site on the patient. The barbed structures 6 can then be utilized to assist in retaining the device 2 within the patient. It is to be understood that the medication is being delivered to the patient during the time period while the barbed structures 6 (and the coating layer 8) degrade. Furthermore, once it has been determined that the device 2 can be removed from the patient (the barbed structures 6 have degraded the desire amount), the delivery of the medication is stopped prior to the device 2 being removed from the patient. The device 2 is then conventionally removed from the patient.

### 3. Advantages of the Invention

Biodegradability of the Coating and the Barbed Structures: The present invention eliminates the need for surgical removal of barbs, thereby reducing trauma and the risk of infection.

Controlled Drug Delivery: The present invention provides precise and localized medication administration with reduced systemic side effects.

Customizable Retention: The present invention allows for adjustable biodegradable barbed structure degradation rates that can be controlled which, in turn, allow clinicians to control the duration of implantation.

Minimized Invasive Removal: A unique aspect of the present invention is that as barbed structures 6 degrade, the device 2 becomes easier to extract, thereby reducing patient discomfort.

While it has not been mentioned, one familiar with the art would realize that the device is not limited by the materials used to create each apparatus that comprises the invention. Any other material type can comprise some or all of the elements in constructing the biodegradable implantable drug delivery device with a barbed structure retention mechanism in various embodiments of the present invention.

Although the present invention has been illustrated and described herein with reference to preferred embodiments and specific examples, it will be readily apparent to those of ordinary skill in the art that other embodiments and examples may perform similar functions and/or achieve like results. All such equivalent embodiments and examples are within the spirit and scope of the present invention, are contemplated thereby, and are intended to be covered by the following claims.

What is claimed is:

1. A biodegradable, implantable drug delivery device, comprising:
a tubular structure having a smooth area located at one end of the tubular structure, wherein the tubular structure is constructed of a non-biodegradable polymer material;
a lumen located within a length of the tubular structure, wherein the lumen is configured to deliver a liquid medication along an interior of the tubular structure to the smooth area; and
a biodegradable coating located on a outer surface of the tubular structure, wherein the biodegradable coating includes a plurality of biodegradable barbed structures formed in the biodegradable coating, wherein the plurality of biodegradable barbed structures is configured to assist in retaining the biodegradable, implantable drug delivery device in place in a patient, and wherein a length and a degradation rate of the plurality of barbed structures are configured to determine a retention period of the device within the patient.

2. The device of claim 1, wherein the non-biodegradable polymer further comprises:
polypropylene;
nylon;
polyester; or
a copolymer thereof.

3. The device of claim 1, wherein the smooth area is further comprised of:
a plurality of openings, wherein the plurality of openings is configured to allow for the delivery of the liquid medication.

4. The device of claim 1, wherein the biodegradable coating and the biodegradable barbed structures are configured to degrade in a controlled manner.

5. The device of claim 1, wherein the device is configured to deliver local anesthetics for short-term pain management.

6. The device of claim 1, wherein the device is configured for long-term drug delivery for chronic pain management.

7. The device of claim 1, wherein the biodegradable, implantable drug delivery device further comprises:
an anti-microbial coating located over the biodegradable coating and the plurality of biodegradable barbed structures.

8. A method of constructing a biodegradable, implantable drug delivery device, comprising:
providing a tubular structure having a smooth area located at one end of the tubular structure, wherein the tubular structure is constructed of a non-biodegradable polymer material;
providing a lumen within a length of the tubular structure, wherein the lumen is configured to deliver a liquid medication along the tubular structure to the smooth area; and
placing a biodegradable coating on a outer surface of the tubular structure, wherein the biodegradable coating includes a plurality of biodegradable barbed structures formed in the biodegradable coating, wherein the plurality of biodegradable barbed structures is configured to assist in retaining the biodegradable, implantable drug delivery device in place in a patient, and wherein a length and a degradation rate of the plurality of barbed structures are configured to determine a retention period of the device within the patient.

9. The method of claim 8, wherein the non-biodegradable polymer further comprises:
polypropylene;
nylon;
polyester; or
a copolymer thereof.

10. The method of claim 8, wherein the smooth area is further comprised of:
a plurality of openings, wherein the plurality of openings is configured to allow for the delivery of the liquid medication.

11. The method of claim 8, wherein the biodegradable coating and the biodegradable barbed structures are configured to degrade in a controlled manner.

12. The method of claim 8, wherein the device is configured to deliver local anesthetics for short-term pain management.

13. The method of claim 8, wherein the device is configured for long-term drug delivery for chronic pain management.

14. The method of claim 7, wherein the biodegradable coating and the biodegradable barbed structures are anti-microbial.

15. A method of using a biodegradable, implantable drug delivery device, comprising:
providing a tubular structure having a smooth area located at one end of the tubular structure, wherein the tubular structure is constructed of a non-biodegradable polymer material;
providing a lumen within a length of the tubular structure, wherein the lumen is configured to deliver a liquid medication along the tubular structure to the smooth area;
placing a biodegradable coating on an outer surface of the tubular structure;
forming a plurality of biodegradable barbed structures in the biodegradable coating, wherein the plurality of biodegradable barbed structures is configured to assist in retaining the biodegradable, implantable drug delivery device in place in a patient, and wherein a length and a degradation rate of the plurality of barbed structures are configured to determine a retention period of the device within the patient;
delivering a medication along the tubular structure to the smooth area, wherein the medication is delivered to the patient;
allowing the biodegradable barbed structures to degrade over a desired period of time and continuing to deliver the medication to the patient;
upon determining that the tubular structure can be removed from the patient, stopping the delivery of the medication; and
removing the tubular structure from the patient.

16. The method of claim 15, wherein the non-biodegradable polymer further comprises:
polypropylene;
nylon;
polyester; or
a copolymer thereof.

17. The method of claim 15, wherein the smooth area is further comprised of:
a plurality of openings, wherein the plurality of openings is configured to allow for the delivery of the liquid medication.

18. The method of claim 15, wherein the biodegradable coating and the biodegradable barbed structures are configured to degrade in a controlled manner.

19. The method of claim 15, wherein the device is configured to deliver local anesthetics for short-term pain management.

20. The method of claim 15, wherein the device is configured for long-term drug delivery for chronic pain management.
